SEQUENCE LISTING (31 sequences)

SEQ ID NO: 1 - protein, 178 aa, Homo sapiens
  SIGNAL 1..18
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 3 - protein, 160 aa, synthetic construct
  MUTAGEN 18: note = N18A
SPGQGTQSENSCTHFPGALPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 4 - protein, 160 aa, synthetic construct
  MUTAGEN 18: note = N18D
SPGQGTQSENSCTHFPGDLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 5 - protein, 160 aa, synthetic construct
  MUTAGEN 18: note = N18M
SPGQGTQSENSCTHFPGMLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 6 - protein, 160 aa, synthetic construct
  MUTAGEN 18: note = N18F
SPGQGTQSENSCTHFPGFLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 7 - protein, 160 aa, synthetic construct
  MUTAGEN 18: note = N18L
SPGQGTQSENSCTHFPGLLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 8 - protein, 160 aa, synthetic construct
SPGQGTQSENSCTHFPGLVPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 9 - protein, 160 aa, synthetic construct
SPGQGTQSENSCTHFPGILPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 10 - protein, 160 aa, synthetic construct
SPGQGTQSENSCTHFPGYLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 11 - protein, 160 aa, synthetic construct
SPGQGTQSENSCTHFPGWLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 12 - protein, 160 aa, synthetic construct
SPGQGTQSENSCTHFPGKLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 13 - protein, 160 aa, synthetic construct
SPGQGTQSENSCTHFPGRLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 14 - protein, 160 aa, synthetic construct
SPGQGTQSENSCTHFPGALPNMLRDLRDAFSRVKTFFQMKDQLDNLLLKESLLEDFKGYLGCQALSEMIQFYLEEVMPQAENQDPDIKAHVNSLGENLKTLRLRLRACHRFLPCENKSKAVEQVKNAFNKLQEKGIYKAMSEFDIFINYIEAYMTMKIRN

SEQ ID NO: 15 - protein, 160 aa, synthetic construct
  MUTAGEN 18: note = N18I
  MUTAGEN 104: note = R104Q
SPGQGTQSENSCTHFPGILPNMLRDLRDAFSRVKTFFQMKDQLDNLLLKESLLEDFKGYLGCQALSEMIQFYLEEVMPQAENQDPDIKAHVNSLGENLKTLRLQLRRCHRFLPCENKSKAVEQVKNAFNKLQEKGIYKAMSEFDIFINYIEAYMTMKIRN

SEQ ID NO: 16 - protein, 160 aa, synthetic construct
  MUTAGEN 18: note = N18Y
  MUTAGEN 104: note = R104Q
SPGQGTQSENSCTHFPGYLPNMLRDLRDAFSRVKTFFQMKDQLDNLLLKESLLEDFKGYLGCQALSEMIQFYLEEVMPQAENQDPDIKAHVNSLGENLKTLRLQLRRCHRFLPCENKSKAVEQVKNAFNKLQEKGIYKAMSEFDIFINYIEAYMTMKIRN

SEQ ID NO: 17 - protein, 160 aa, synthetic construct
  MUTAGEN 18: note = N18Y
  MUTAGEN 104: note = R104Q
  MUTAGEN 107: note = R107A
SPGQGTQSENSCTHFPGYLPNMLRDLRDAFSRVKTFFQMKDQLDNLLLKESLLEDFKGYLGCQALSEMIQFYLEEVMPQAENQDPDIKAHVNSLGENLKTLRLQLRACHRFLPCENKSKAVEQVKNAFNKLQEKGIYKAMSEFDIFINYIEAYMTMKIRN

SEQ ID NO: 18 - protein, 406 aa, synthetic construct
  DOMAIN 161..175: note = linker
  DOMAIN 176..406: note = Fc
  DOMAIN 1..160: note = N18A
SPGQGTQSENSCTHFPGALPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGGSGGGGSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 5 aa, synthetic construct
  REGION 1..5: note = L1
  REPEAT 1..5: note = 1-6 repeats
GGGGS

SEQ ID NO: 20 - protein, 5 aa, synthetic construct
  REGION 1..5: note = L2
  REPEAT 1..5: note = 1-6 repeats
SSSSG

SEQ ID NO: 21 - skipped

SEQ ID NO: 22 - protein, 5 aa, synthetic construct
EAAAK

SEQ ID NO: 23 - skipped

SEQ ID NO: 24 - protein, 6 aa, synthetic construct
  REGION 1..6: note = L6
  REPEAT 1..6: note = 1-6 repeats
  SITE 6: note = MISC_FEATURE - X is G or S
NLYFQX

SEQ ID NO: 25 - protein, 20 aa, synthetic construct
MYRMQLLSCIALSLALVTNS

SEQ ID NO: 26 - protein, 447 aa, synthetic construct
EVQLVESGGGLVQPGGSLRLSCAASGFTIDNTYIHWVRQAPGKGLEWVARIDPANGMTRYADSVKGRFMISADTSKNTAYLQMNSLRAEDTAVYYCARAFSWFPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGA

SEQ ID NO: 27 - protein, 214 aa, synthetic construct
DIQMTQSPSSLSASVGDRVTITCRAGQDVYKAVAWYQQKPGKAPKLLIYWSTNLYSGVPSRFSGSGSGTDFTLTISSLQPEDFATYYCQQGSTWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 443 aa, synthetic construct
EVQLVESGGGLVQPGGSLRLSCAASGFTIDNTYIHWVRQAPGKGLEWVARIDPANGMTRYADSVKGRFMISADTSKNTAYLQMNSLRAEDTAVYYCARAFSWFPDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 29 - protein, 214 aa, synthetic construct; identical to SEQ ID NO: 27

SEQ ID NO: 30 - protein, 450 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 216 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

(19) United States
(12) Patent Application Publication
(10) Pub. No.: US 2024/0190934 A1
(43) Pub. Date: Jun. 13, 2024

(54) ENGINEERED INTERLEUKIN-10 AND FUSION PROTEINS THEREOF

(71) Applicant: Elixiron Immunotherapeutics (Hong Kong) Limited, Central Hong Kong (CN)

(72) Inventors: Hung-Kai Chen, Lost Altos, CA (US); Po-Hao Chang, New Taipei City (TW); Wei Huang, Taipei City (TW); Jing-Yi Huang, Taipei City (TW)

(21) Appl. No.: 18/519,188

(22) Filed: Nov. 27, 2023

Related U.S. Application Data
(60) Provisional application No. 63/385,130, filed on Nov. 28, 2022.

(51) Int. Cl. C07K 14/54; A61K 38/00; A61K 45/06; A61K 47/68; C07K 16/28

(52) U.S. Cl. CPC C07K 14/5428; A61K 45/06; A61K 47/6803; C07K 16/2827; A61K 38/00; C07K 2317/24; C07K 2317/31; C07K 2319/00

(57) ABSTRACT

The present disclosure provides an IL-10 variant protein, a fusion protein comprising the IL-10 variant protein and a polypeptide, and the use thereof. The present disclosure also provides a method of producing the same and a pharmaceutical composition comprising the same.

### CROSS-REFERENCE TO RELATED APPLICATIONS

This application claims the benefit of U.S. Provisional Application No. 63/385,130, filed Nov. 28, 2022, the content of which is herein incorporated by reference in its entirety.

### SEQUENCE LISTING STATEMENT

The contents of the electronic sequence listing titled 23P0592-seq list.xml (Size: 39,626 bytes; and Date of Creation: Nov. 23, 2023) is herein incorporated by reference in its entirety.

### FIELD OF INVENTION

The present disclosure relates to an IL-10 variant protein, a fusion protein, and the use thereof. The present disclosure further relates a method of producing the IL-10 variant protein or the fusion protein.

### BACKGROUND OF THE INVENTION

IL-10 is a key immunomodulatory cytokine with therapeutic potentials. Binding of IL-10 to IL-10 receptor results in the activation of three STAT transcription factors: STAT1, STAT3, and STAT5. The IL-10/STAT3 axis contributes to anti-inflammatory and immune tolerance1. IL-10 exhibits anti-tumor activity through activation of intra-tumoral cytotoxic CD8+ T cells^{2,3}. IL-10 can bind to the IL-10 receptor on tumor-resident CD8+ T cells and directly activate and expand tumor-resident CD8+ T Cells⁴. Administration of PEGylated IL-10 (Pegilodecakin) induced system immune activation in cancer patients, including elevated Granzyme B and IFNγ levels and sustained expansion of activated CD8+ T cells in both blood and tumor⁵.

Systemic IL-10 therapy has been investigated in clinical trials, but the efficacy was complicated by short half-life and systemic toxicities. Different approaches, including oral administration of IL-10 recombinant protein or fusion of IL-10 to a targeting antibody to prevent systemic toxicity have been investigated^{6,7,8}. Local delivery of IL-10 could also be achieved by local administration of plasmid DNA, virus, mRNA or using IL-10-transduced host cells. For example, Tumor infiltrating lymphocytes (TILs), Tregs or chimeric antigen receptor (CAR) T-cell which will be homing to tumor site. Engineered TILs or CART cells expressing IL-10 could provide autocrine signaling to enhance effector functions and proliferation of T cells. For these approaches, IL-10 variant proteins with greater CD8 stimulating activity are in need.

There have been attempts to generate IL-10 variant proteins with higher binding affinity to IL-10 receptor by yeast display system^{9,10,11}. An engineered monomeric IL-10 variant protein (M1) was used in the yeast display because wide-type IL-10 could not be promptly expressed on yeast surface and IL-10 variant proteins with enhanced IL-10 receptor binding were identified. Theses variants are with multiple substitutions and show enhanced STAT3 activation in some cell types but not others. For example, IL-10 variant protein Super 10 with four amino acid substitutions (N18Y, N92Q, T100D, R104W) induced enhanced STAT1 but not STAT3 activity in CD4+ or CD8+ T cells¹¹. And the ability of Super 10 to induce Granzyme B secretion in CD8+ T cells was significantly diminished when compared to wild-type IL-10. Different screening strategy for IL-10 variant proteins will influence outcome.

### SUMMARY OF THE INVENTION

The present disclosure relates to new IL-10 variant proteins and IL-10 variant fusion molecules with modified CD8 stimulating activity. The IL-10 variant proteins may be administered, e.g. via plasmid, virus or RNA, directly at tumor site, leading to local expression of the IL-10 variant proteins in the tumor tissues so as to avoid the systemic toxicity. The IL-10 variant proteins may also be administered using IL-10 variant-transduced host cells. In addition, the fusion protein with the IL-10 variant proteins, e.g. IL-10-Fc or antibody-IL-10, may be used in the preparation of biologics.

To achieve the above propose, one aspect of the present disclosure provided herein is an IL-10 variant protein, including: (1) a single substitution of amino acid at position 18, relative to amino acids of wild-type IL-10; (2) a first substitution of amino acid at position 18 and a second substitution of amino acid at position 104 or position 107, relative to amino acids of wild-type IL-10; or (3) a first substitution of amino acid at position 18, a second substitution of amino acid at position 104 and a third substitution of amino acid at position 107, relative to amino acids of wild-type IL-10.

Preferably, the wild-type IL-10 includes the amino acid sequence having at least 80%, preferably at least 90%, at least 95%, at least 98%, and more preferably at least 99%, identity with SEQ ID NO: 2.

Preferably, the substitution of amino acid at position 18 includes N18A, N18D, N18I, N18Y, N18M, N18F, N18L, N18W, N18K or N18R.

Preferably, the substitution of amino acid at position 104 includes R104Q.

Preferably, the substitution of amino acid at position 107 includes R107A, R107E, R107Q or R107D.

Preferably, the IL-10 variant protein further includes at least one substitution selected from the group consisting of N18A/R104Q, N18A/R107A, N18A/R107E, N18A/R107Q, N18A/R107D, N18D/R104Q, N18D/R107A, N18D/R107E, N18D/R107Q, N18D/R107D, N18M/R104Q, N18M/R107A, N18M/R107E, N18M/R107Q, N18M/R107D, N18F/R104Q, N18F/R107A, N18F/R107E, N18F/R107Q, N18F/R107D, N18L/R104Q, N18L/R107A, N18L/R107E, N18L/R107Q, N18L/R107D, N18A/R104Q/R107A, N18A/R104Q/R107E, N18A/R104Q/R107Q, N18A/R104Q/R107D, N18D/R104Q/R107A, N18D/R104Q/R107E, N18D/R104Q/R107Q, N18D/R104Q/R107D, N18M/R104Q/R107A, N18M/R104Q/R107E, N18M/R104Q/R107Q, N18M/R104Q/R107D, N18F/R104Q/R107A, N18F/R104Q/R107E, N18F/R104Q/R107Q, N18F/R104Q/R107D, N18L/R104Q/R107A, N18L/R104Q/R107E, N18L/R104Q/R107Q, N18L/R104Q/R107D, N18W/R104Q/R107A, N18W/R104Q/R107E, N18W/R104Q/R107Q, N18W/R104Q/R107D, N18K/R104Q/R107A, N18K/R104Q/R107E, N18K/R104Q/R107Q, N18K/R104Q/R107D, N18R/R104Q/R107A, N18R/R104Q/R107E, N18R/R104Q/R107Q, and N18R/R104Q/R107D.

Preferably, the IL-10 variant protein is monomer or dimer.

Preferably, the IL-10 variant protein further includes a signal peptide.

Preferably, the signal peptide includes amino acid sequence of SEQ ID NO: 25.

In another aspect of the present disclosure, provided herein is a fusion protein, including: (1) a polypeptide, wherein the polypeptide includes an antibody or a fragment thereof, an antagonist, a receptor or a ligand of the target protein, a half-life extension moiety, or a protein-Trap; and (2) an aforementioned IL-10 variant protein fused to the polypeptide.

Preferably, the polypeptide are fused to the IL-10 variant protein via a linker.

Preferably, the linker includes the amino acid sequence of SEQ ID NO: 19-24.

Preferably, the IL-10 variant protein is fused to N-terminal or C-terminal of the polypeptide.

Preferably, the IL-10 variant protein is monomer or dimer.

Preferably, the fusion protein further includes an amino acid sequence of SEQ ID NO: 18.

Preferably, the antibody includes an anti-PD-L1 antibody.

Preferably,
the IL-10 variant protein includes:
   the amino acid sequence having at least 80%, preferably at least 90%, and more preferably at least 95%, identity with SEQ ID NO: 14; and the anti-PD-L1 antibody includes:
   YP7G IgG1 comprising the heavy chain amino acid sequence having at least 80%, preferably at least 90%, and more preferably at least 95%, identity with SEQ ID NO: 26 and the light chain amino acid sequence having at least 80%, preferably at least 90%, and more preferably at least 95%, identity with SEQ ID NO: 27;
   YP7G IgG4 comprising the heavy chain amino acid sequence having at least 80%, preferably at least 90%, and more preferably at least 95%, identity with SEQ ID NO: 28 and the light chain amino acid sequence having at least 80%, preferably at least 90%, and more preferably at least 95%, identity with SEQ ID NO: 29; or
   Avelumab comprising the heavy chain amino acid sequence having at least 80%, preferably at least 90%, and more preferably at least 95%, identity with SEQ ID NO: 30 and the light chain amino acid sequence having at least 80%, preferably at least 90%, and more preferably at least 95%, identity with SEQ ID NO: 31.

Preferably, (1) the antibody is a human, humanized, or chimeric antibody; (2) the antibody is a full length antibody of class IgG, optionally, wherein the class IgG antibody has an isotype selected from IgG1, IgG2, IgG3, and IgG4; (3) the antibody includes an Fc region variant, optionally an Fc region variant that alters effector function and/or a variant that alters antibody half-life; (4) the antibody is an antibody fragment, optionally selected from the group consisting of F(ab′)2, Fab′, Fab, Fv, single domain antibody (VHH), and scFv; (5) the antibody includes an immunoconjugate, optionally, wherein the immunoconjugate includes a therapeutic agent for treatment of a CSFIR-mediated, PDL1-mediated, PD1-mediated or VEGF-mediated disease or condition; or (6) the antibody is a multi-specific antibody, optionally a bispecific antibody.

In another aspect of the present disclosure, provided herein is an isolated polynucleotide or vector encoding the aforementioned IL-10 variant protein or the aforementioned fusion protein.

In another aspect of the present disclosure, provided herein is an isolated host cell including the isolated polynucleotide or aforementioned vector.

In another aspect of the present disclosure, provided herein is a method of producing an IL-10 variant protein or a fusion protein, including culturing the aforementioned host cell so that an IL-10 variant protein or a fusion protein is produced.

In another aspect of the present disclosure, provided herein is a pharmaceutical composition including the aforementioned IL-10 variant protein, the aforementioned fusion protein, or the aforementioned isolated polynucleotide or aforementioned vector, optionally together with a pharmaceutically acceptable carrier, diluent or excipient.

In another aspect of the present disclosure, provided herein is a use of the aforementioned IL-10 variant protein, the aforementioned fusion protein or the aforementioned isolated polynucleotide or vector, for the manufacture of a medicament.

### BRIEF DESCRIPTION OF THE DRAWINGS

FIG. 1 depicts the STAT3 activity induced by IL-10 variant proteins in a cell-based screening using HeLa IL-10R1-STAT3 luciferase reporter cells. HeLa IL-10R1-STAT3 reporter cells were transfected with plasmids expressing wild-type IL-10 or its variants. The luciferase signals were measured at 48 hours post transfection. Data shown are percentage changes in relative luminescence unit (RLU) compared to wild-type IL-10 plasmid transfection.

FIG. 2 depicts the dose-dependent activation of STAT3 by IL-10 containing culture supernatant in HeLa IL-10R1-STAT3 reporter cells. IL-10 containing culture supernatants were collected from wild-type HeLa cells transfected with increasing amounts of plasmids (0-60 ng/well) encoding wild-type IL-10 or variants. The luciferase signals were measured at 16 hours after addition of the IL-10 containing culture supernatant to HeLa IL-10R1-STAT3 reporter cells.

FIG. 3 depicts the ability of N18A and N18D to better enhance Granzyme B secretion from CD8+ T cell when compared to wild-type IL-10, Super 10 and R5A11. IL-10 containing culture supernatant collected from IL-10 plasmid-transfected HeLa cells was used to treat CD8+ T cells.

FIG. 4 depicts that the purified recombinant IL-10 variant proteins, N18A and N18D, exhibited higher STAT3 activation activity in HeLa IL-10R1-STAT3 reporter cells when compared to wild-type (WT) IL-10. All data are shown as a fold change of relative luminescence unit (RLU) when compared to WT and is the average of the indicated number of independent biological replicates +SD, **p≤0.01, ***p≤ 0.001.

FIG. 5 depicts that the purified IL-10 variant proteins including N18A, N18F, N18I, N18L and N18Y, induced higher granzyme B secretion from CD8+ T cells when compared to wild-type IL-10.

FIG. 6A depicts the purification yield of IL-10-Fc fusion proteins. Addition of R104Q or R104Q/R107A substitution to IL-10 (N18 variant)-Fc fusion proteins increased protein purification yield.

FIG. 6B depicts the purification yield of IL-10-Fc fusion proteins. Addition of R104Q or R104Q/R107A substitution to IL-10 (N18 variant)-Fc fusion proteins increased protein purification yield.

FIG. 6C depicts the purification yield of IL-10-Fc fusion proteins. Addition of R104Q or R104Q/R107A substitution to IL-10 (N18 variant)-Fc fusion proteins increased protein purification yield.

FIG. 7A depicts the results of STAT3 activation in HeLa IL-10R1-STAT3 reporter cells induced by IL-10-Fc fusion proteins. (A) IL-10 (N18I)-Fc and IL-10 (N18I/R104Q)-Fc induced higher STAT3 activation when compared to wild-type IL-10 (WT)-Fc fusion protein. (B) IL-10 (N18Y)-Fc, IL-10 (N18Y/R104Q) and IL-10 (N18/R104Q/R107A)-Fc induced higher STAT3 activation when compared to wild-type IL-10 (WT)-Fc fusion protein.

FIG. 7B depicts the results of STAT3 activation in HeLa IL-10R1-STAT3 reporter cells induced by IL-10-Fc fusion proteins. (A) IL-10 (N18I)-Fc and IL-10 (N18I/R104Q)-Fc induced higher STAT3 activation when compared to wild-type IL-10 (WT)-Fc fusion protein. (B) IL-10 (N18Y)-Fc, IL-10 (N18Y/R104Q) and IL-10 (N18I/R104Q/R107A)-Fc induced higher STAT3 activation when compared to wild-type IL-10 (WT)-Fc fusion protein.

FIG. 8A depicts the result of STAT3 activation in HeLa IL-10R1-STAT3 reporter cells induced by Avelumab-IL-10 (wild-type), Avelumab-IL-10 (R107A) and Avelumab-IL-10 (N18A/R107A) fusion proteins. Addition of N18A substitution to Avelumab-IL-10 (R107A) resulted in higher Granzyme B secretion from CD8+ T cells. FIG. 8B depicts the purification yield of Avelumab-IL-10 fusion proteins. Data are presented as mean±SD, *P<0.05 (n=3).

FIG. 9A depicts the result of dose-dependent Granzyme B secretion from CD8+ T cells treated with YP7G-IL-10 (R107A) and YP7G-IL-10 (N18A/R107A) fusion proteins in (A) IgG1 format and (B) IgG4 format. Addition of N18A substitution to YP7G-IL-10 (R107A) resulted in higher Granzyme B secretion from CD8+ T cells.

FIG. 9B depicts the result of dose-dependent Granzyme B secretion from CD8+ T cells treated with YP7G-IL-10 (R107A) and YP7G-IL-10 (N18A/R107A) fusion proteins in

(A) IgG1 format and (B) IgG4 format. Addition of N18A substitution to YP7G-IL-10 (R107A) resulted in higher Granzyme B secretion from CD8+ T cells.

### DETAILED DESCRIPTION

The foregoing and other aspects of the present disclosure will now be described in more detail with respect to other embodiments described herein. It should be appreciated that the invention can be embodied in different forms and should not be construed as limited to the embodiments set forth herein. Rather, these embodiments are provided so that this disclosure will be thorough and complete and fully convey the invention's scope to those skilled in the art.

The terminology used in the description of the invention herein is for the purpose of describing particular embodiments only and is not intended to be limiting to the invention. As used in the description of the invention and the appended claims, the singular forms “a”, “an” and “the” are intended to include the plural forms as well, unless the context clearly indicates otherwise.

As used herein, the terms “comprises,” “comprising,” “includes,” “including,” “has,” “having,” “contains”, “containing,” or any other variation thereof, are intended to cover a non-exclusive inclusion, subject to any limitation explicitly indicated. For example, a composition, mixture, process or method that includes a list of elements is not necessarily limited to only those elements but may include other elements not expressly listed or inherent to such composition, mixture, process, or method.

As used herein, the term “about” indicates that a value includes, for example, the inherent variation of error for a measuring device, the method employed to determine the value, or the variation among the study subjects. Typically the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

The use of the term “or” in the claims is used to mean “and/or” unless explicitly indicated to refer to alternatives only or the alternatives are mutually exclusive. However, the disclosure supports a definition that refers to only alternatives and “and/or.”

Unless otherwise defined, all technical and scientific terms used herein have the same meaning as commonly understood by one of ordinary skill in the art to which this invention belongs. All publications, patent disclosure, patents, and other references cited herein are incorporated by reference in their entirety for the teachings relevant to the sentence and/or paragraph in which the reference is presented.

The following representative examples illustrate various features and embodiments of the disclosure, which are intended to be illustrative and not limiting. Those skilled in the art will readily appreciate that the specific examples are only illustrative of the invention as described more fully in the subsequent claims. Every embodiment and feature described in the present application should be understood to be interchangeable and combinable with every embodiment contained within.

The propose of this disclosure is to provide an IL-10 variant protein, including: (1) a single substitution of amino acid at position 18, relative to amino acids of wild-type IL-10; (2) a first substitution of amino acid at position 18 and a second substitution of amino acid at position 104 or position 107, relative to amino acids of wild-type IL-10; or (3) a first substitution of amino acid at position 18, a second substitution of amino acid at position 104 and a third substitution of amino acid at position 107, relative to amino acids of wild-type IL-10.

In an embodiment, the wild-type IL-10 includes the amino acid sequence having at least 80%, preferably at least 90%, at least 95%, at least 98%, and more preferably at least 99%, identity with SEQ ID NO: 2.

The term “IL-10 variant protein” refers to the wild-type IL-10 with 1, 2, 3, or more substitutions of amino acid within its sequence. The substitution numberings for the IL-10 variant protein are based on the wild-type IL-10 sequence, e.g. SEQ ID NO: 2. The term “wild-type IL-10” refers to a naturally occurring IL-10 without artificial mutations. The wild-type IL-10 from various organisms include the following sequences referenced from NCBI accession numbers: 1INR_A (human), NP_001129092.2 (chimpanzee), XP_004028338.1 (gorilla), and ABI63906.1 (rhesus macaque), XP_046539742.1 (zebra), XP_044614621.1 (assess), XP_006094927.1 (little brown bat), XP_058394138.1 (rhinoceros).

In an embodiment, the substitution of amino acid at position 18 includes N18A, N18D, N18I, N18Y, N18M, N18F, N18L, N18W, N18K or N18R. The substitution of amino acid at position 104 includes R104Q. The substitution of amino acid at position 107 includes R107A, R107E, R107Q or R107D.

In an embodiment, the IL-10 variant protein further includes at least one substitution selected from the group consisting of N18A/R104Q, N18A/R107A, N18A/R107E, N18A/R107Q, N18A/R107D, N18D/R104Q, N18D/R107A, N18D/R107E, N18D/R107Q, N18D/R107D, N18M/R104Q, N18M/R107A, N18M/R107E, N18M/R107Q, N18M/R107D, N18F/R104Q, N18F/R107A, N18F/R107E, N18F/R107Q, N18F/R107D, N18L/R104Q, N18L/R107A, N18L/R107E, N18L/R107Q, N18L/R107D, N18A/R104Q/R107A, N18A/R104Q/R107E, N18A/R104Q/R107Q, N18A/R104Q/R107D, N18D/R104Q/R107A, N18D/R104Q/R107E, N18D/R104Q/R107Q, N18D/R104Q/R107D, N18M/R104Q/R107A, N18M/R104Q/R107E, N18M/R104Q/R107Q, N18M/R104Q/R107D, N18F/R104Q/R107A, N18F/R104Q/R107E, N18F/R104Q/R107Q, N18F/R104Q/R107D, N18L/R104Q/R107A, N18L/R104Q/R107E, N18L/R104Q/R107Q, N18L/R104Q/R107D, N18W/R104Q/R107A, N18W/R104Q/R107E, N18W/R104Q/R107Q, N18W/R104Q/R107D, N18K/R104Q/R107A, N18K/R104Q/R107E, N18K/R104Q/R107Q, N18K/R104Q/R107D, N18R/R104Q/R107A, N18R/R104Q/R107E, N18R/R104Q/R107Q, and N18R/R104Q/R107D.

The term “substitution” refers to the replacement of one or more amino acids with other amino acids in a protein sequence. For example, N18A depicts that the original amino acid at position 18 in a protein sequence, asparagine (N), is replaced with alanine (A). Similarly, R107D depicts that the original amino acid at position 107 in a sequence, arginine (R), is replaced with aspartic acid (D). The substitution representation of (original amino acid)(position)(replaced amino acid) may consistent with the commonly used in the art.

In an embodiment, the IL-10 variant protein further includes a signal peptide. Preferably, the signal peptide includes amino acid sequence of SEQ ID NO: 25.

The term “signal peptide” refers to a short sequence that is typically attached to the N-terminus of a protein, and is responsible for directing the protein to their correct destination within the cell, including the endoplasmic reticulum, Golgi apparatus, or cell membrane. The signal peptide is mainly 10 to 30 amino acids long and includes three domains: (1) N-terminal domain, consisting of positively charged amino acids, binds to translocate proteins. Translocate proteins help transport the signal peptide and the protein across the membrane.; (2) Hydrophobic core domain forms a stable a-helix structure that helps the signal peptide insert into the membrane; (3) C-terminal domain includes a recognition site for signal peptidase, which is an enzyme that cleaves the signal peptide from the protein after it has crossed the membrane. As the signal peptide helps to transport and localize the protein after translation, it is crucial for improving the production of proteins. Preferably, the signal peptide includes amino acid sequence of MYRMQLLSCIALSLALVTNS (SEQ ID NO: 25).

The present disclosure also provides a fusion protein, including: (1) a polypeptide, wherein the polypeptide includes an antibody or a fragment thereof, an antagonist, a receptor or a ligand of the target protein, a half-life extension moiety, or a protein-Trap; and (2) an aforementioned IL-10 variant protein fused to the polypeptide.

The term “antibody” refers to a molecule comprising one or more polypeptide chains that specifically binds to, or is immunologically reactive with, a particular antigen. Exemplary antibodies of the present disclosure include monoclonal antibodies, polyclonal antibodies, chimeric antibodies, humanized antibodies, human antibodies, antibody fusions (e.g., fusion proteins), multispecific antibodies (i.e., bispecific antibodies), monovalent antibodies (e.g., single-arm antibodies), multivalent antibodies, antigen-binding fragments (e.g., Fab′, F(ab′)₂, Fab, Fv, rIgG, and scFv fragments), and synthetic antibodies (or antibody mimetics).

The term “full-length antibody” refers to an antibody having a structure substantially similar to a native antibody structure or having heavy chains that contain an Fc region as defined herein.

The term “antibody fragment” refers to a portion of a full-length antibody which is capable of binding the same antigen as the full-length antibody. Examples of antibody fragments include, but are not limited to, Fv, Fab, Fab′, Fab′-SH, F(ab′)₂; diabodies; linear antibodies; monovalent, or single-armed antibodies; single-chain antibody molecules (e.g., scFv); and multispecific antibodies formed from antibody fragments.

The term “antagonist” refers to a molecule that binds to a receptor and prevents the receptor from binding to its ligand. The antagonist may be mainly classified into two types: competitive antagonist and non-competitive antagonist. Competitive antagonist binds to the same binding site as the receptor, and preventing the receptor from binding to its ligand. Non-competitive antagonist binds to a different binding site on the receptor, thereby altering the receptor's activity by changing its structure. Exemplary antagonists of the present disclosure include Bevacizumab, Ranibizumab, Brolucizumab, Sevacizumab, Varisacumab, Navicixizumab, Vulinacimab, Olinvacimab, Alacizumab, Icrucumab, Aflibercept, Conbercept, or anti-PD-L1 IgG1 antibody fragments (Atezolizumab, Avelumab, or Durvalumab).

The term “half-life extension moiety” refers to a molecule that can increase the half-life of a protein. The moiety could maintain the fusion protein's stability or enzyme activity. Exemplary half-life extension moieties of the present disclosure include XTEN protein polymers¹², GlycoTAIL and FlexiTAIL¹³, Albumin-binding domain¹⁴, PASylation¹⁵, ELPylation¹⁶, HAPylation¹⁷, or Gelatin-like protein¹⁸.

The term “protein-Trap” refers to a molecule that allows for the identification of proteins of interest based on their unique subcellular localization without the need for specific antibodies to each protein. Protein-Trap may tag proteins with an epitope, and then use specific antibodies against the epitope to localize the tagged proteins. This allows the identification of proteins that are localized to specific subcellular compartments, such as the nucleus, cytoplasm, or membrane. Exemplary protein-Traps of the present disclosure include epitope-tag developed by Sineshchekova et al.¹⁹, or GFP-tag²⁰,

In an embodiment, the IL-10 variant protein may be fused to a polypeptide via a linker.

The terms “peptide linker” or “linker” refer to a peptide of 1-42 amino acids that may increase protein stability or folding, protein expression, improve biological activity, or enable targeting to the protein. A wide range of polypeptide linkers are known in the art and could be used in the compositions and methods of the present disclosure, including A(EAAAK)₄ALEA(EAAAK)₄A, (GGGGS)_{n=1,2,4,} PAPAP, AEAAAKEAAAKA, (AP)ₙ₌₅₋₁₇. In a preferred embodiment, the peptide linker that may be used in the present disclosure are listed below.

| | | |
|---|---|---|
| Linker 1 | (GGGGS)n; n = 1-6 | SEQ ID NO: 19 |
| | repeat(s) | |
| Linker 2 | (SSSSG)n; n = 1-6 | SEQ ID NO: 20 |
| | repeat(s) | |
| Linker 3 | (G)n; n = 1-6 repeat(s) | SEQ ID NO: 21 |
| Linker 4 | (EAAAK)n; n = 1-6 | SEQ ID NO: 22 |
| | repeat(s) | |
| Linker 5 | (XP)n; n = 1-6 repeat(s) | SEQ ID NO: 23 |
| Linker 6 | ENLYFQ(-G/S); n = 1-6 | SEQ ID NO: 24 |
| | repeat(s) | |

In an embodiment, the IL-10 variant protein is fused to N-terminal or C-terminal of the polypeptide.

The terms “N-terminal” or “C-terminal” refer to the two ends of a protein. The N-terminal is the end of the chain that contains the free amino group, while the C-terminal is the end of the chain that contains the free carboxyl group. The protein are typically written from N-terminal to C-terminal in the art.

In an embodiment, the IL-10 variant protein is monomer or dimer.

The terms “monomer” or “dimer” refer to the structure of a protein. A monomer refers to a single protein molecule, while a dimer refers to a protein molecule made up of two monomer units that are held together by weak or noncovalent interactions, such as hydrogen bonds or hydrophobic interactions. The dimer protein could be either homodimer or heterodimer. Homodimer is made up of two identical monomer units, and Heterodimer is made up of two different monomer units. In the present disclosure, IL-10 is typically a homodimer in the organism. The dimeric structure of IL-10 allows it to bind to two receptors, which enhances its signaling ability. Still, IL-10 can exist in monomer form, but the monomer is less active.

In an embodiment, the fusion protein further includes an amino acid sequence of SEQ ID NO: 18.

In an embodiment, the antibody includes an anti-PD-L1 antibody.

The term “anti-PD-L1 antibody” refers to an antibody that binds PD-L1 with sufficient affinity such that the antibody is useful as a therapeutic and/or diagnostic agent for targeting PD-L1.

In an embodiment, the IL-10 variant protein comprises: the amino acid sequence having at least 80%, preferably at least 90%, and more preferably at least 95%, identity with SEQ ID NO: 14; and the anti-PD-L1 antibody comprises: YP7G IgGI comprising the heavy chain amino acid sequence having at least 80%, preferably at least 90%, and more preferably at least 95%, identity with SEQ ID NO: 26 and the light chain amino acid sequence having at least 80%, preferably at least 90%, and more preferably at least 95%, identity with SEQ ID NO: 27; YP7G IgG4 comprising the heavy chain amino acid sequence having at least 80%, preferably at least 90%, and more preferably at least 95%, identity with SEQ ID NO: 28 and the light chain amino acid sequence having at least 80%, preferably at least 90%, and more preferably at least 95%, identity with SEQ ID NO: 29; or Avelumab comprising the heavy chain amino acid sequence having at least 80%, preferably at least 90%, and more preferably at least 95%, identity with SEQ ID NO: 30 and the light chain amino acid sequence having at least 80%, preferably at least 90%, and more preferably at least 95%, identity with SEQ ID NO: 31.

In an embodiment, (1) the antibody is a human, humanized, or chimeric antibody; (2) the antibody is a full length antibody of class IgG, optionally, wherein the class IgG antibody has an isotype selected from IgG1, IgG2, IgG3, and IgG4; (3) the antibody includes an Fc region variant, optionally an Fc region variant that alters effector function and/or a variant that alters antibody half-life; (4) the antibody is an antibody fragment, optionally selected from the group consisting of F(ab′)2, Fab′, Fab, Fv, single domain antibody (VHH), and scFv; (5) the antibody includes an immunoconjugate, optionally, wherein the immunoconjugate includes a therapeutic agent for treatment of a CSFIR-mediated, PDL1-mediated, PD1-mediated or VEGF-mediated disease or condition; or (6) the antibody is a multi-specific antibody, optionally a bispecific antibody.

The term “human antibody” refers to an antibody which possesses an amino acid sequence corresponding to that of an antibody produced by a human or a human cell or derived from a nonhuman source that utilizes human antibody repertoires or other human antibody-encoding sequences. This definition of a human antibody specifically excludes a humanized antibody including non-human antigen-binding residues.

The term “humanized antibody” refers to a chimeric antibody comprising amino acid sequences from non-human HVRs and amino acid sequences from human FRs. In certain embodiments, a humanized antibody will include substantially all of at least one, and typically two, variable domains, in which all or substantially all of the HVRs correspond to those of a non-human antibody, and all or substantially all of the FRs correspond to those of a human antibody. A humanized antibody optionally may include at least a portion of an antibody constant region derived from a human antibody. A “humanized form” of an antibody, e.g., a non-human antibody, refers to an antibody that has undergone humanization.

The term “chimeric antibody” refers to an antibody in which a portion of the heavy and/or light chain is derived from a particular source or organisms, while the remainder of the heavy and/or light chain is derived from a different source or organisms.

The terms “hypervariable region” or “HVR” refer to each of the regions of an antibody variable domain which are hypervariable in sequence and/or form structurally defined loops (“hypervariable loops”). Generally, native antibodies comprise four chains with six HVRs; three in the heavy chain variable domain, VH (HVR-H1, HVR-H2, HVR-H3), and three in the light chain variable domain, V_{L} (HVR-L1, HVR-L2, HVR-L3). The HVRs generally comprise amino acid residues from the hypervariable loops and/or from the “complementarity determining regions” (CDRs).

The term “FR” refers to variable domain residues other than the HVR residues. The FRs of a variable domain generally consist of four domains: FRI, FR2, FR3, and FR4. Accordingly, the HVR and FR sequences generally appear in the following sequence in VH (or VL): FR1-H1(L1)-FR2-H2(L2)-FR3-H3(L3)-FR4.

The term “class” of an antibody refers to the type of constant domain or constant region possessed by its heavy chain. There are five major classes of antibodies: IgA, IgD, IgE, IgG, and IgM, and several of these are further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

The term “Fc region” refers to a dimer complex including the C-terminal polypeptide sequences of an immunoglobulin heavy chain, wherein a C-terminal polypeptide sequence is that which is obtainable by papain digestion of an intact antibody. The Fc region may comprise native or variant Fc sequences. Although the boundaries of the Fc sequence of an immunoglobulin heavy chain may vary, the human IgG heavy chain Fc sequence is usually defined to stretch from an amino acid residue at about position Cys226, or from about position Pro230, to the carboxyl-terminus of the Fc sequence. However, the C-terminal lysine (Lys447) of the Fc sequence may or may not be present. The Fc sequence of an immunoglobulin generally comprises two constant domains, a CH2 domain and a CH3 domain, and optionally comprises a CH4 domain.

The term “Fc region variant” refers to the Fc region sequence (e.g., a human IgG1, IgG2, IgG3, or IgG4 Fc region) including an amino acid substitution at one or more amino acid residue positions.

The term “F(ab′)₂” refers to a pair of Fab fragments which are generally covalently linked near their carboxy termini by hinge cysteines between them. Other chemical couplings of antibody fragments also are known in the art.

The term “Fab” refers to an antibody fragment that contains the VH, CHI and VL, CL regions, linked by an intramolecular disulfide bond.

The term “Fab” refers to an antibody fragment that contains the Fab fragment with a free sulfhydryl group on CH1. It may be alkylated or conjugated with an enzyme, toxin, or other protein of interest.

The term “Fv” refers to an antibody fragment which contains a complete antigen recognition and binding site. This region consists of a dimer of one heavy and one light chain variable domain in tight association, which can be covalent in nature, for example in scFv. It is in this configuration that the three HVRs of each variable domain interact to define an antigen binding site on the surface of the VH-VL dimer. Collectively, the six HVRs or a subset thereof confer antigen binding specificity to the antibody. However, even a single variable domain (or half of an Fv comprising only three HVRs specific for an antigen) has the ability to recognize and bind antigen, although usually at a lower affinity than the entire binding site.

The term “single domain antibody (VHH)” refers to a fragment of an antibody consisting of the variable domain of the heavy chain (VH). Canonical antibodies include two heavy chains and two light chains, while single-domain antibodies only have one heavy chain.

The terms “Single-chain Fv” or “scFv” refer to antibody fragments including the VH and VL domains of an antibody, wherein these domains are present in a single polypeptide chain. Generally, an Fv polypeptide further includes a polypeptide linker between the VH and VL domains which enables the scFv to form the desired antigen binding structure.

The term “multi-specific antibody” refers to an antibody that has at least two different binding sites, and each with a binding specificity for a different antigen. Optionally, the term “bispecific antibody” refers to an antibody that has two different binding sites.

The present disclosure also provides an isolated polynucleotide or vector encoding the aforementioned IL-10 variant protein or the aforementioned fusion protein.

The present disclosure also provides an isolated host cell comprising the aforementioned isolated polynucleotide or aforementioned vector.

The term “polynucleotide” refers to a biopolymer composed of nucleotide monomers covalently bonded in a chain. The example of polynucleotide includes DNA (deoxyribonucleic acid), RNA (ribonucleic acid), mRNA (messenger RNA) and analogs of the DNA or RNA generated using nucleotide analogs. The polynucleotide may have an open reading frame encoding an antigenic polypeptide and may be used as a ribonucleic acid vaccine (NAV). Once the polynucleotide is delivered to the body including injection, inhalation, or oral administration, it may be taken up by cells and translated into the antigenic polypeptide, which is then presented to the immune system, triggering an immune response. Exemplary polynucleotides as NAVs of the present disclosure include the IL-10 variant proteins and the fusion proteins thereof.

The term “vector” refers to a molecule that carries genetic material (e.g., polynucleotide) into another cell, and may be used for protein expression, gene therapy, vaccine development, and genetic engineering. The vector may be delivered to the body as the NAV. In the present disclosure, one or more vectors that encode the antigenic polypeptide are provided. Exemplary vectors of the present disclosure include adenovirus, retrovirus, poxvirus, adeno-associated virus, baculovirus, herpes simplex virus, pcDNA3.4, pET, pCMV, pUC19, pBR322 pUC18, POET, or pMV261.

The term “isolated host cell” refers to a cell that could produce the protein encoded by a polynucleotide or vector, such as E. coli. For example, recombinant production of an IL-10 variant protein or its fusion protein is carried out by isolating a nucleic acid encoding the IL-10 variant protein or its fusion protein. The nucleic acid is then inserted into one or more vectors for further cloning and/or expression in the isolated host cell. Suitable isolated host cells and culturing methods for cloning or expressing the protein-encoding vectors are well-known in the art and include prokaryotic or eukaryotic cells. After expression, the protein may be isolated from cell paste in a soluble fraction and further purified.

The present disclosure also provides a method of producing an IL-10 variant protein or a fusion protein, comprising culturing the aforementioned host cell so that an IL-10 variant protein or a fusion protein is produced.

The present disclosure also provides a pharmaceutical composition comprising the aforementioned IL-10 variant protein, the aforementioned fusion protein, or the aforementioned isolated polynucleotide or aforementioned vector, optionally together with a pharmaceutically acceptable carrier, diluent or excipient.

The term “pharmaceutical composition” refers to a preparation in a form that allows the biological activity of the active ingredient(s) to be effective, and which contain no additional components which are toxic to the subjects to which the composition is administered. A pharmaceutical composition may include one or more active agents. For example, a pharmaceutical composition may include an anti-PD-L1 antibody as the sole active agent of the formulation or may include an anti-PD-L1 antibody and one or more additional active agents, an immune activator such as IL-10, or an inhibitor of an immune checkpoint molecule.

The term “pharmaceutically acceptable carrier” refers to an ingredient in a pharmaceutical composition, other than an active ingredient, which is nontoxic to the subject to whom it is administered. A pharmaceutically acceptable carrier includes a buffer, excipient, stabilizer, or preservative.

The present disclosure also provides a use of the aforementioned IL-10 variant protein, the aforementioned fusion protein or the aforementioned isolated polynucleotide or aforementioned vector, for the manufacture of a medicament.

### EXAMPLES

The following examples are given merely to illustrate the present disclosure and not in any way to limit its scope.

### Example 1: Design of IL-10 Variant Proteins

Sequences for illustrative IL-10 wild-type (WT) and variants comprising substitutions are depicted below. It should be noted that the substitution numberings for IL-10 monomer are based on the mature form IL-10 sequence, i.e. SEQ ID NO: 2.

IL-10 precursor sequence
(SEQ ID NO: 1)
MHSSALLCCL VLLTGVRASP GQGTQSENSC THFPGNLPNM LRDLRDAFSR
VKTFFQMKDQ LDNLLLKESL LEDFKGYLGC QALSEMIQFY LEEVMPQAEN
QDPDIKAHVN SLGENLKTLR LRLRRCHRFL PCENKSKAVE QVKNAFNKLQ
EKGIYKAMSE FDIFINYIEA YMTMKIRN
IL-10 mature form sequence
(SEQ ID NO: 2)

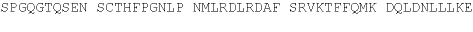

SLLEDFKGYL GCQALSEMIQ FYLEEVMPQA ENQDPDIKAH VNSLGENLKT
LRLRLRRCHR FLPCENKSKA VEQVKNAFNK LQEKGIYKAM SEFDIFINYI
EAYMTMKIRN
IL-10 N18A
(SEQ ID NO: 3)

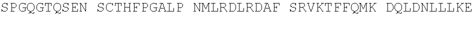

SLLEDFKGYL GCQALSEMIQ FYLEEVMPQA ENQDPDIKAH VNSLGENLKT
LRLRLRRCHR FLPCENKSKA VEQVKNAFNK LQEKGIYKAM SEFDIFINYI
EAYMTMKIRN
IL-10 N18D
(SEQ ID NO: 4)

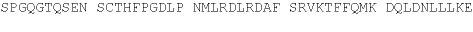

SLLEDFKGYL GCQALSEMIQ FYLEEVMPQA ENQDPDIKAY VNSLGENLKT
LRLRLRRCHR FLPCENKSKA VEQVKNAFNK LQEKGIYKAM SEFDIFINYI
EAYMTMKIRN
IL-10 N18M
(SEQ ID NO: 5)

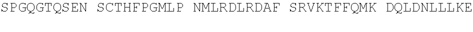

SLLEDFKGYL GCQALSEMIQ FYLEEVMPQA ENQDPDIKAH VNSLGENLKT
LRLRLRRCHR FLPCENKSKA VEQVKNAFNK LQEKGIYKAM SEFDIFINYI
EAYMTMKIRN
IL-10 N18F
(SEQ ID NO: 6)

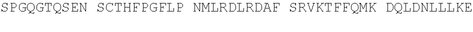

SLLEDFKGYL GCQALSEMIQ FYLEEVMPQA ENQDPDIKAH VNSLGENLKT
LRLRLRRCHR FLPCENKSKA VEQVKNAFNK LQEKGIYKAM SEFDIFINYI
EAYMTMKIRN
IL-10 N18L
(SEQ ID NO: 7)

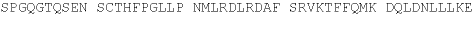

SLLEDFKGYL GCQALSEMIQ FYLEEVMPQA ENQDPDIKAH VNSLGENLKT
LRLRLRRCHR FLPCENKSKA VEQVKNAFNK LQEKGIYKAM SEFDIFINYI
EAYMTMKIRN
IL-10 N18V
(SEQ ID NO: 8)

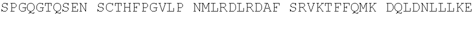

SLLEDFKGYL GCQALSEMIQ FYLEEVMPQA ENQDPDIKAH VNSLGENLKT
LRLRLRRCHR FLPCENKSKA VEQVKNAFNK LQEKGIYKAM SEFDIFINYI
EAYMTMKIRN
IL-10 N18I
(SEQ ID NO: 9)

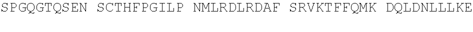

SLLEDFKGYL GCQALSEMIQ FYLEEVMPQA ENQDPDIKAH VNSSLGENLKT
LRLRLRRCHR FLPCENKSKA VEQVKNAFNK LQEKGIYKAM SEFDIFINYI
EAYMTMKIRN
IL-10 N18Y
(SEQ ID NO: 10)

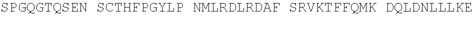

SLLEDFKGYL GCQALSEMIQ FYLEEVMPQA ENQDPDIKAH VNSLGENLKT
LRLRLRRCHR FLPCENKSKA VEQVKNAFNK LQEKGIYKAM SEFDIFINYI
EAYMTMKIRN
IL-10 N18W
(SEQ ID NO: 11)

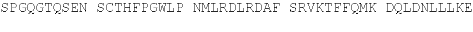

SLLEDFKGYL GCQALSEMIQ FYLEEVMPQA ENQDPDIKAH VNSLGENLKT
LRLRLRRCHR FLPCENKSKA VEQVKNAFNK LQEKGIYKAM SEFDIFINYI
EAYMTMKIRN
IL-10 N18K
(SEQ ID NO: 12)

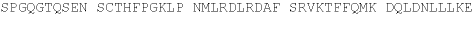

SLLEDFKGYL GCQALSEMIQ FYLEEVMPQA ENQDPDIKAH VNSLGENLKT
LRLRLRRCHR FLPCENKSKA VEQVKNAFNK LQEKGIYKAM SEFDIFINYI
EAYMTMKIRN
IL-10 N18R
(SEQ ID NO: 13)

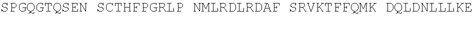

SLLEDFKGYL GCQALSEMIQ FYLEEVMPQA ENQDPDIKAH VNSLGENLKT
LRLRLRRCHR FLPCENKSKA VEQVKNAFNK LQEKGIYKAM SEFDIFINYI
EAYMTMKIRN
IL-10 N18A/R107A
(SEQ ID NO: 14)

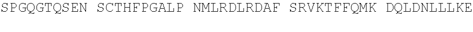

SLLEDFKGYL GCQALSEMIQ FYLEEVMPQA ENQDPDIKAH VNSLGENLKT

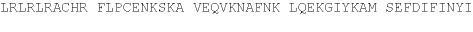

EAYMTMKIRN
IL-10 N18I/R104Q
(SEQ ID NO: 15)

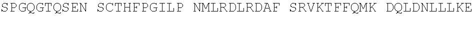

SLLEDFKGYL GCQALSEMIQ FYLEEVMPQA ENQDPDIKAH VNSLGENLKT

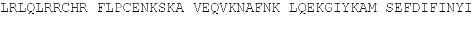

EAYMTMKIRN
IL-10 N18Y/R104Q
(SEQ ID NO: 16)

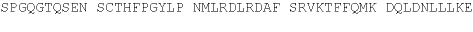

SLLEDFKGYL GCQALSEMIQ FYLEEVMPQA ENQDPDIKAH VNSLGENLKT

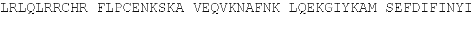

EAYMTMKIRN
IL-10 N18Y/R104Q/R107A
(SEQ ID NO: 17)

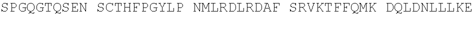

SLLEDFKGYL GCQALSEMIQ FYLEEVMPQA ENQDPDIKAH VNSLGENLKT

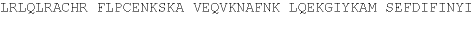

EAYMTMKIRN
IL-10 N18A-Fc
(SEQ ID NO: 18)
SPGQGTQSENSCTHFPGALPNMLRDLRDAFSRVKTFFQMKDQLDNL
LLKESLLEDFKGYLGCQALSEMIQFYLEEVMPQAENQDPDIKAHVNSL
GENLKTLRLRLRRCHRFLPCENKSKAVEQVKNAKFNKLQEKGIYKAMSEF
DIFINYIEAYMTMKIRNGGGGSGGGGSGGGGSPKSCDKTHTCPPCPA
PELLGGPSVFLFPPKPKDTLMISRTPEVTCVVVDVSHEDPEVKFNWY
VDGVEVHNAKTKPREEQYNSTRYVVSVLTVLHQDWLNGKEYKCKVS
NKALPAPIEKTISKAKGQPREPQVYTLPPSRDELTKNQVSLTCLVKGFY
PSDIAVEWESNGQPENNYKTTPPVLKSDGSFFLYSKLTVDKSRWQQ
GNVFSCSVMHEALHNHYTQKSLSLSPGK

IL-10 sequence is italicized, linker is underlined, human IgG Fc is in bold.

In an embodiment, the IL-10 variant protein of the present disclosure does not include at least one amino acid substitution at positions 92 and/or 99. In a preferred embodiment, the IL-10 variant protein of the present disclosure does not include the group of amino acid substitutions at positions 18, 92 and 99. In a preferred embodiment, the IL-10 variant protein of the present disclosure does not include the group of amino acid substitutions at positions 18, 92, 99, and 111.

In another embodiment, the IL-10 variant protein of the present disclosure does not include at least one amino acid substitution at positions 14, 21, 24, 25, 28, 74, 90, 92, 96, and/or 100. In a preferred embodiment, the IL-10 variant protein of the present disclosure does not include the group of amino acid substitutions at positions 18, 21, 25, 92, 96, 100, and 104. In a preferred embodiment, the IL-10 variant protein of the present disclosure does not include the group of amino acid substitutions at positions 14, 18, 21, 24, 25, 28, 74, 90, 92, 96, 100 and 104.

### Example 2: Screening of IL-10 Variant Protein by Transient Transfection of IL-10 Variant Encoding Plasmid into a Stable Reporter Cell Line

Local delivery of IL-10 using mRNA, viruses, DNA vectors, or administration of host cells engineered with IL-10 expression has the potential to avoid adverse effects induced by IL-10 systemic administration. IL-10 will be expressed and secreted by cells at disease sites or by IL-10 transduced host cells traveling to disease sites. The secreted IL-10 in the target environment could act in autocrine or paracrine fashion and activate cells expressing IL-10 receptor which is composed of IL-10R1 and IL-10R2. Screening assays using phage display or yeast display mainly identify IL-10 variant proteins based on binding ability of IL-10 to IL-10 receptor rather than activation of specific signaling pathways. Change of binding affinity to IL-10 receptor is not necessarily associated with change of a specific activity on target cells due to the complexity of receptor signaling. To simulate the expression and function of IL-10 at the site of action, a cell-based assay was established, and the assay could identify IL-10 variant proteins with either changed activity, changed expression level or both.

To establish the cell-based screening assay, HeLa cells were chosen because it can be easily transfected (for the cell-based screening). HeLa cells express endogenous IL-10R2 but not IL-10R1. Therefore, HeLa IL-10R1-STAT3 luciferase reporter cells which stably expressing human IL-10R1 and a STAT3 Firefly luciferase reporter gene under the transcriptional control of the STAT3 were generated. STAT3 Firefly luciferase reporter lentivirus was purchased from Cellomics Technology (PLV-10065-50). Lentivirus encoding IL-10R1 (NM_001558) was generated using transfer vector pLAS5w.Phyg and standard method. Puromycin and hygromycin were used for selection of HeLa cells expressing both IL-10R1 and STAT3 reporter gene. The resulting stable HeLa IL-10R1-STAT3 cells were used for subsequent screening and activity studies. Transfection of HeLa IL-10R1-STAT3 cells with IL-10 expressing plasmid will result in IL-10 protein secretion and the secreted IL-10 protein could in turn bind to the cell surface IL-10 receptor (IL-10R1/IL-10R2) and hence the induction of STAT3-dependent luciferase activity.

Expression vector pcDNA3.4 containing the IL-2 signal peptide (MYRMQLLSCIALSLALVTNS, SEQ ID NO: 25) followed by sequence of IL-10 wild-type (SEQ ID NO: 2) or its variants (e.g. SEQ ID NO: 3-17) were constructed by GenScript services.

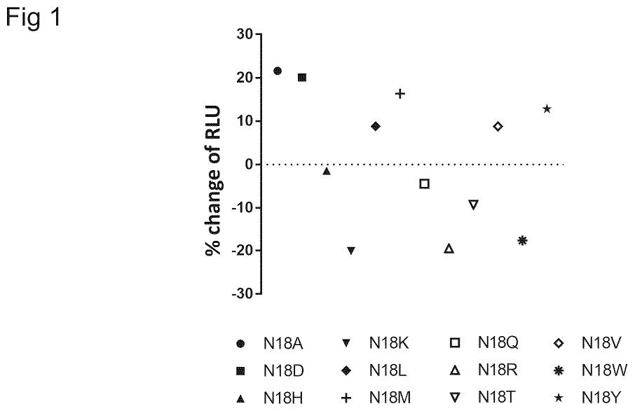

The Hela IL-10R1-STAT3 cells were grown at 37° C. with 5% CO2 using DMEM medium supplemented with 10% FBS and 1% Pen/Strep. One day prior to transfection, HeLa IL-10R1-STAT3 cells were seeded into a 96 well solid white flat bottom TC treated plate in 100 μl of growth medium at 2 x 104 cells/well. Transient transfection of HeLa IL-10R1-STAT3 cells with 1.25 ng/well pcDNA3.4 plasmid expressing wild-type IL-10 or variants was performed using PolyJet transfection reagent (SignaGen Laboratories: SL100688). After 6 hours, medium was removed and replaced with fresh medium. At 48 hours post transfection, luciferase activity was determined by ONE-Glo™ Luciferase Assay System (Promega: E6120) according to the manufacturer's instructions. The relative light units (RLU) of luciferase activity obtained with wild-type IL-10 plasmid transfection was set as basal. STAT3 activation activity of IL-10 variant protein was expressed as a percentage increase or decrease over basal activity. Amino acid substitutions of asparagine at position 18 (N18) of IL-10 lead to various impacts on STAT3 activation. Substitution of N18 with amino acids alanine (N18A), aspartic acid (N18D), leucine (N18L), methionine (N18M), valine (N18V) and tyrosine (Y) resulted in increased STAT3 activity while substitution of N18 with lysine (N18K), arginine (N18R) and tryptophan (W) resulted in significantly decreased STAT3 activation (FIG. 1).

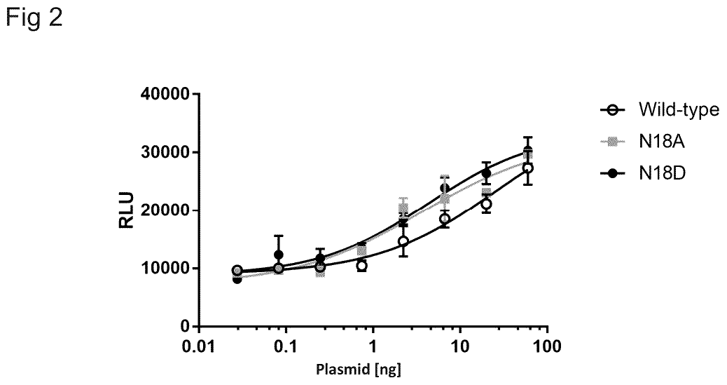

To confirm that the secreted IL-10 variant protein has the same effects on STAT3 activation when work as a paracrine, the same HeLa IL-10R1-STAT3 reporter cells was used but the source of IL-10 protein was replaced with culture supernatant harvested from transfection of wild-type IL-10 or variants into wild-type HeLa cells. First, IL-10 containing culture supernatant was generated as followed. Wild-type HeLa cells were seeded into a solid-bottom 96-well microplate in 100 μl of growth medium at 3×104 cells/well. HeLa cells were transfected with different amounts of plasmids expressing wild-type IL-10 or variants (ranging from 60 ng/well to 0.0275 ng/well generated with 3-fold dilution) with Polyjet according to the vendor's protocol. Then, the supernatants containing secreted IL-10 protein were collected after 48 hours and then added into the culture of HeLa IL-10R1-STAT3 reporter cells. After culturing for another 16 hours, luciferase activity was determined by ONE-Glo™ Luciferase Assay System according to the manufacturer's instructions. Culture supernatant from transfection of IL-10 variant proteins N18A and N18D induced higher STAT3 activation when compared to wild-type IL-10 (FIG. 2) across a wide range of amounts of plasmids used for transfection.

### Example 3: CD8+ T Stimulating Activity

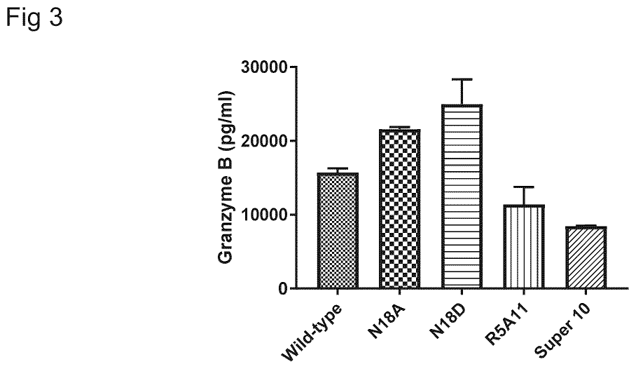

Granzyme B plays crucial roles in anti-tumor activity of CD8+ T cells, and IL-10 can improve production of granzyme B both in vitro and in vivo through STAT3 activation. The effect of amino acid substitution at N18 on Granzyme B secretion was investigated. CD8+ T cells were isolated from PBMC using Ficoll-Paque PLUS (Cytiva: 17144003) and purified with CD8 microbeads (Miltenyi Biotec: 130-045-201). After stimulating for 72 hours with T cell TransAct (Miltenyi Biotec: 130-111-160) in AIM-V medium (Thermo Fisher Scientific: 12055083), activated CD8+ T cells were rested for 4 hours. Culture supernatants collected from wild-type HeLa cells transfected with wild-type IL-10 or variants (as previous described) were added to CD8+ T cells culture. After another 72 hours culture at 37° C. in a CO₂ incubator, culture supernatants of the IL-10 treated CD8+ T cell were collected, and the concentration of Granzyme B were determined by ELISA (R&D systems: DY2906-05) according to the manufacturer's instructions. Treatment of CD8+ T cells with culture supernatant containing N18A or N18D induced more Granzyme B secretion from CD8+ T cells when compared to culture supernatant containing wild-type IL-10 or IL-10 variant proteins R5A11 or Super 10 (FIG. 3). R5A11 (WO2021181091A1) and Super 10 (WO2021243057A1) are IL-10 variant proteins with higher binding affinity to IL-10 receptor.

In summary, single amino acid substitution at N18 of IL-10 could modulate IL-10 activity.

### Example 4: STAT3 Activation by Purified IL-10 Variant Proteins

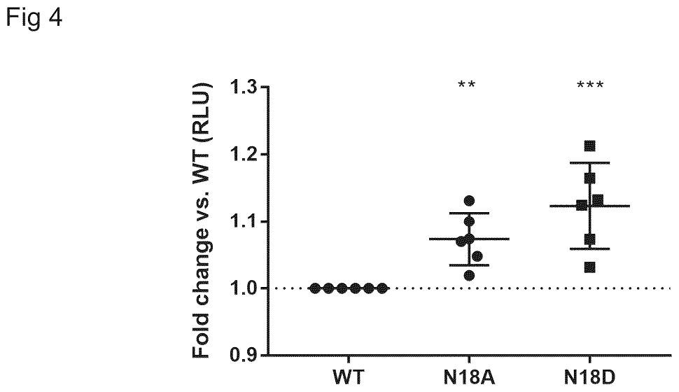

The changed IL-10 activity in culture supernatant containing IL-10 variant proteins, could be due to change in protein expression level or protein activity or both. The ability of purified IL-10 variant proteins to activate STAT3 was investigated. Wild-type IL-10 and N18 variants were cloned into pcDNA3.4 with a N-terminal IL-2 signal peptide and a C-terminal (G4S)x3 linker plus a poly-histidine tag (6×His) for purification. Proteins were produced by transient transfection in F293 cells following the manufacturer's protocol (ThermoFisher FreeStyle 293 Expression System) and then purified by standard Ni-NTA chromatography. The purity of purified proteins was confirmed by SDS-PAGE and commissive blue. Activity of purified wild-type IL-10 and variants was assessed in HeLa IL-10R1-STAT3 cells. HeLa IL-10R1-STAT3 cells were seeded into a white solid-bottom 96-well microplate in 100 μl of growth medium at 5×104 cells/well. Cells were then incubated at 37° C. in a CO₂ incubator for another 6 hours or overnight for cells to attach to the plate and then the cells were stimulated with 8 ng/ml or 24 ng/ml of His-tagged wild-type IL-10 or variants. After another 18 hours culture, STAT3 luciferase activity was determined by ONE-Glo™ Luciferase Assay System (Promega E6120) according to the manufacturer's instructions. As show in FIG. 4, purified IL-10 variant proteins, N18A and N18D, showed higher STAT3 activation activity when compared to wild-type IL-10.

### Example 5: IL-10 Fc Fusion Proteins

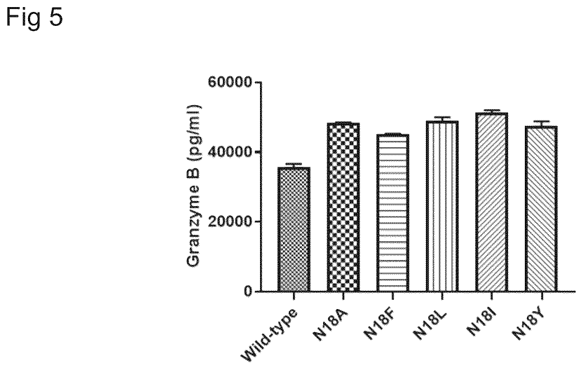

Amino acid substitution at position 18 was introduced into IL-10-Fc fusion protein by standard mutagenesis techniques. Proteins were produced by transient transfection in CHO-S cells and purified by a two-step purification process comprising protein A chromatography and SEC chromatography. CD8+ T cell treatment and Granzyme B measurement was performed as described in Example 2. As shown in FIG. 5, IL-10-Fc fusion proteins with IL-10 variant proteins N18A, N18F, N18I, N18L and N18Y exhibited higher CD8+ T cells stimulating activity.

### Example 6: Additional Engineering

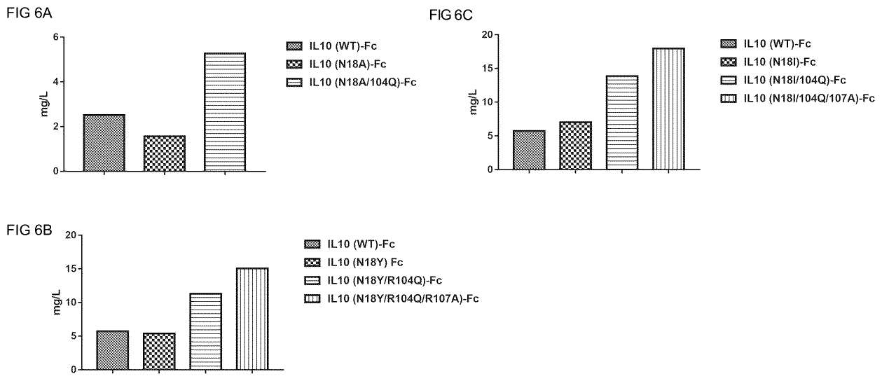

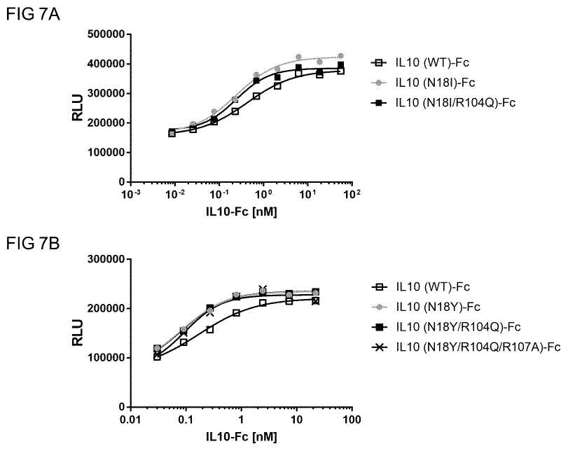

In our owned patent application (PCT/US22/77660), we have shown that substitution of arginine at amino acid 104, 107 or both (especially R104Q, R107A or combination) of IL-10 improves the purification yield of IL-10 fusion proteins. If the property of improved manufacturability and enhanced activity could be combined into one molecule, it would benefit the development of IL-10 fusion biologics. In the exemplary experiments, N18 substitution was introduced into IL-10 (R104Q)-Fc and IL-10 (R104Q/R107A)-Fc fusion proteins by standard mutagenesis techniques. Proteins were produced by transient transfection in CHO-S cells and purified by a two-step purification process comprising protein A chromatography and SEC chromatography. When N18A substitution was introduced into IL-10 (R104Q)-Fc fusion protein, the resulting IL-10 (N18A/R104Q)-Fc fusion protein had better purification yield when compared to IL-10 (WT)-Fc or IL-10 (N18A)-Fc fusion proteins (FIG. 6A). When N18I substitution was introduced into IL-10 (R104Q)-Fc and IL-10 (R104Q/R107A)-Fc fusion protein, the resulting IL-10 (N18I/R104Q)-Fc and IL-10 (N18I/R104Q/R107A)-Fc had better purification yield when compared to IL-10 (WT)-Fc or IL-10 (N18I)-Fc fusion proteins (FIG. 6B). When N18Y substitution was introduced into IL-10 (R104Q)-Fc and IL-10 (R104Q/R107A)-Fc fusion protein, the resulting IL-10 (N18Y/R104Q)-Fc and IL-10 (N18Y/R104Q/R107A)-Fc had better purification yield when compared to IL-10 (WT)-Fc or IL-10 (N18Y)-Fc fusion proteins (FIG. 6C). The activity of the new variants was evaluated using HeLa IL-10R1-STAT3 cells. The activity of IL-10 (N18I/R104Q)-Fc is comparable to IL-10 (N18I)-Fc and is better than IL-10 (WT)-Fc (FIG. 7A). The activity of IL-10 (N18Y/R104Q)-Fc or IL-10 (N18Y/R104Q/R107A)-Fc is comparable to IL-10 (N18Y)-Fc and is better than IL-10 (WT)-Fc (FIG. 7B).

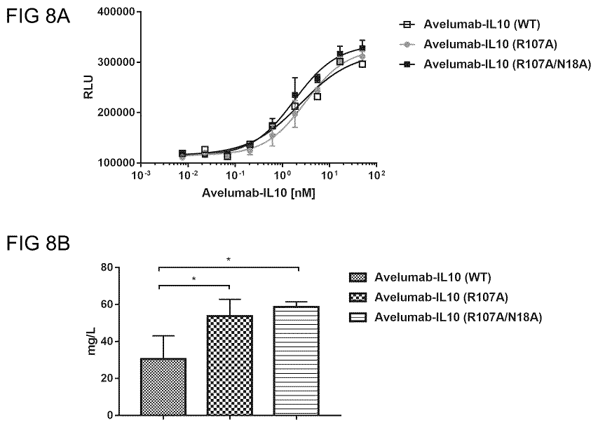

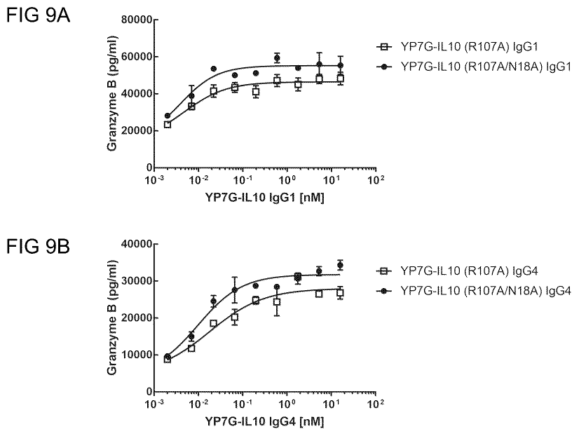

The benefit of combining these substitutions were also tested in antibody-IL-10 fusion format. N18A substitution was introduced into antibody-IL-10 fusion proteins by standard mutagenesis techniques. Details of exemplary antibody-IL-10 fusion proteins including YP7G-IL-10 (R107A) and Avelumab-IL-10 (R107A) could be found in WO2021231741 and PCT/US22/77660. Proteins were produced by transient transfection in CHO-S cells and purified by a two-step purification process comprising protein A chromatography and SEC chromatography. Introduction of N18A substitution to Avelumab-IL-10 (R107A) significantly enhanced the STAT3 activation and maintained the improved yield by R107A substitution (FIG. 8). Addition of the N18A substitution to YP7G-IL-10(R107A) either on IgG1 (HC: SEQ ID NO: 26; LC: SEQ ID NO: 27) or IgG4 (HC: SEQ ID NO: 28; LC: SEQ ID NO: 29) format also significantly enhanced the Granzyme B secretion from CD8+ T cells (FIG. 9).

### REFERENCES

The references listed below and referred to herein are hereby incorporated into this specification by reference unless this specification expressly provides otherwise.
1. SARAIVA, Margarida; VIEIRA, Paulo; O'GARRA, Anne. Biology and therapeutic potential of interleukin-10. Journal of Experimental Medicine, 2019, 217.1: e20190418.
2. MUMM, John B., et al. IL-10 elicits IFNγ-dependent tumor immune surveillance. Cancer cell, 2011, 20.6: 781-796.
3. FUJII, Shin-ichiro, et al. Interleukin-10 promotes the maintenance of antitumor CD8+ T-cell effector function in situ. Blood, The Journal of the American Society of Hematology, 2001, 98.7: 2143-2151.
4. EMMERICH, Jan, et al. IL-10 directly activates and expands tumor-resident CD8+ T cells without de novo infiltration from secondary lymphoid organs. Cancer research, 2012, 72.14:3570-3581.
5. NAING, Aung, et al. PEGylated IL-10 (Pegilodecakin) induces systemic immune activation, CD8+ T cell invigoration and polyclonal T cell expansion in cancer patients. Cancer Cell, 2018, 34.5: 775-791. e3.
6. FAY, Nicole C., et al. A novel fusion of IL-10 engineered to traffic across intestinal epithelium to treat colitis. The Journal of Immunology, 2020, 205.11: 3191-3204.
7. BRUIJNEN, Stefan T G, et al. F8-IL10: a new potential antirheumatic drug evaluated by a PET-guided translational approach. Molecular pharmaceutics, 2018, 16.1: 273-281.
8. PELLERIN, Laurence, et al. APVO210: a bispecific anti-CD86-IL-10 fusion protein (ADAPTIR™) to induce antigen-specific T regulatory type 1 cells. Frontiers in immunology, 2018, 9: 881.
9. JOSEPHSON, Kristopher, et al. Design and analysis of an engineered human interleukin-10 monomer. Journal of Biological Chemistry, 2000, 275.18: 13552-13557.
10. GORBY, Claire, et al. Engineered IL-10 variants elicit potent immunomodulatory effects at low ligand doses. Science signaling, 2020, 13.649: eabc0653.
11. SAXTON, Robert A., et al. Structure-based decoupling of the pro-and anti-inflammatory functions of interleukin-10. Science, 2021, 371.6535: eabc8433.
12. PODUST, Vladimir N., et al. Extension of in vivo half-life of biologically active molecules by XTEN protein polymers. Journal of Controlled Release, 2016, 240: 52-66.
13. SEIFERT, Oliver; KONTERMANN, Roland E. GlycoTAIL and FlexiTAIL as half-life extension modules for recombinant antibody fragments. Molecules, 2022, 27.10: 3272.
14. TAN, Huanbo, et al. Albumin-binding domain extends half-life of glucagon-like peptide-1. European Journal of Pharmacology, 2021, 890: 173650.
15. SCHLAPSCHY, Martin, et al. PASylation: a biological alternative to PEGylation for extending the plasma half-life of pharmaceutically active proteins. Protein Engineering, Design & Selection, 2013, 26.8: 489-501.
16. FLOSS, Doreen M., et al. Elastin-like polypeptides revolutionize recombinant protein expression and their biomedical application. Trends in biotechnology, 2010, 28.1: 37-45.
17. ANAND, Resmi; VALLOORAN, Jijo. Polypeptides: PASylation and XTEN. In: Engineering of biomaterials for drug delivery systems. Woodhead Publishing, 2018. p. 299-315.
18. BINDER, Uli; SKERRA, Arne. Half-life extension of therapeutic proteins via genetic fusion to recombinant PEG mimetics. Therapeutic protein—strategies to modulate their plasma half-lives. Wiley-VCH, Weinheim, 2012.
19. SINESHCHEKOVA, Olga O., et al. Protein-trap version 2.1: screening for expressed proteins in mammalian cells based on their localizations. BMC Cell Biology, 2004, 5.1: 1-10.
20. DING, Da-Qiao, et al. Large-scale screening of intracellular protein localization in living fission yeast cells by the use of a GFP-fusion genomic DNA library. Genes to Cells, 2000, 5.3: 169-190.

1. An IL-10 variant protein, comprising:
(1) a single substitution of amino acid at position 18, relative to amino acids of wild-type IL-10;
(2) a first substitution of amino acid at position 18 and a second substitution of amino acid at position 104 or position 107, relative to amino acids of wild-type IL-10; or (3) a first substitution of amino acid at position 18, a second substitution of amino acid at position 104 and a third substitution of amino acid at position 107, relative to amino acids of wild-type IL-10.

2. The IL-10 variant protein according to claim 1, wherein the wild-type IL-10 comprises the amino acid sequence having at least 80%, preferably at least 90%, at least 95%, at least 98%, and more preferably at least 99%, identity with SEQ ID NO: 2.

3. The IL-10 variant protein according to claim 1, wherein the substitution of amino acid at position 18 comprises N18A, N18D, N18I, N18Y, N18M, N18F, N18L, N18W, N18K or N18R.

4. The IL-10 variant protein according to claim 1, wherein the substitution of amino acid at position 104 comprises R104Q.

5. The IL-10 variant protein according to claim 1, wherein the substitution of amino acid at position 107 comprises R107A, R107E, R107Q or R107D.

6. The IL-10 variant protein according to claim 1, comprising at least one substitution selected from the group consisting of N18A/R104Q, N18A/R107A, N18A/R107E, N18A/R107Q, N18A/R107D, N18D/R104Q, N18D/R107A, N18D/R107E, N18D/R107Q, N18D/R107D, N18M/R104Q, N18M/R 107A, N18M/R107E, N18M/R107Q, N18M/R107D, N18F/R104Q, N18F/R107A, N18F/R107E, N18F/R107Q, N18F/R107D, N18L/R104Q, N18L/R107A, N18L/R107E, N18L/R107Q, N18L/R107D, N18A/R104Q/R107A, N18A/R104Q/R107E, N18A/R104Q/R107Q, N18A/R104Q/R107D, N18D/R104Q/R107A, N18D/R104Q/R107E, N18D/R104Q/R107Q, N18D/R104Q/R107D, N18M/R104Q/R107A, N18M/R104Q/R107E, N18M/R104Q/R107Q, N18M/R104Q/R107D, N18F/R104Q/R107A, N18F/R104Q/R107E, N18F/R104Q/R107Q, N18F/R104Q/R107D, N18L/R104Q/R107A, N18L/R104Q/R107D, N18W/R104Q/R107Q, N18L/R104Q/R107E, N18L/R104Q/R107Q, N18W/R104Q/R107E, N18K/R104Q/R107A, N18W/R104Q/R107A, N18W/R104Q/R107D, N18K/R104Q/R107E, N18K/R104Q/R107Q, N18K/R104Q/R107D, N18R/R104Q/R107A, N18R/R104Q/R107D. N18R/R104Q/R107E, N18R/R104Q/R107Q, and N18R/R104Q/R107D.

7. The IL-10 variant protein according to claim 1, wherein the IL-10 variant protein is monomer or dimer.

8. The IL-10 variant protein according to claim 1, further comprising a signal peptide.

9. The IL-10 variant protein according to claim 8, wherein the signal peptide comprises amino acid sequence of SEQ ID NO: 25.

10. A fusion protein, comprising:
(1) a polypeptide, wherein the polypeptide comprises an antibody or a fragment thereof, an antagonist, a receptor or a ligand of the target protein, a half-life extension moiety, or a protein-Trap; and
(2) an IL-10 variant protein according to claim 1 fused to the polypeptide.

11. The fusion protein according to claim 10, wherein the polypeptide are fused to the IL-10 variant protein via a linker.

12. The fusion protein according to claim 11, wherein the linker comprises the amino acid sequence of SEQ ID NO: 19-24.

13. The fusion protein according to claim 10, wherein the IL-10 variant protein is fused to N-terminal or C-terminal of the polypeptide.

14. The fusion protein according to claim 10, wherein the IL-10 variant protein is monomer or dimer.

15. The fusion protein according to claim 10, further comprising an amino acid sequence of SEQ ID NO: 18.

16. The fusion protein according to claim 10, wherein the antibody comprises an anti-PD-L1 antibody.

17. The fusion protein according to claim 16, wherein
the IL-10 variant protein comprises:
the amino acid sequence having at least 80%, preferably at least 90%, and more preferably at least 95%, identity with SEQ ID NO: 14; and
the anti-PD-L1 antibody comprises:
YP7G IgG1 comprising the heavy chain amino acid sequence having at least 80%, preferably at least 90%, and more preferably at least 95%, identity with SEQ ID NO: 26 and the light chain amino acid sequence having at least 80%, preferably at least 90%, and more preferably at least 95%, identity with SEQ ID NO: 27;
YP7G IgG4 comprising the heavy chain amino acid sequence having at least 80%, preferably at least 90%, and more preferably at least 95%, identity with SEQ ID NO: 28 and the light chain amino acid sequence having at least 80%, preferably at least 90%, and more preferably at least 95%, identity with SEQ ID NO: 29; or Avelumab comprising the heavy chain amino acid sequence having at least 80%, preferably at least 90%, and more preferably at least 95%, identity with SEQ ID NO: 30 and the light chain amino acid sequence having at least 80%, preferably at least 90%, and more preferably at least 95%, identity with SEQ ID NO: 31.

18. The fusion protein according to claim 10, wherein:
(1) the antibody is a human, humanized, or chimeric antibody;
(2) the antibody is a full length antibody of class IgG, optionally, wherein the class IgG antibody has an isotype selected from IgG1, IgG2, IgG3, and IgG4;
(3) the antibody comprises an Fc region variant, optionally an Fc region variant that alters effector function and/or a variant that alters antibody half-life;
(4) the antibody is an antibody fragment, optionally selected from the group consisting of F(ab′)2, Fab′, Fab, Fv, single domain antibody (VHH), and scFv;
(5) the antibody comprises an immunoconjugate, optionally, wherein the immunoconjugate comprises a therapeutic agent for treatment of a CSFIR-mediated.
